(19) United States
(12) Patent Application Publication
(10) Pub. No.: US 2021/0196184 A1
(43) Pub. Date: Jul. 1, 2021

(54) NON-INVASIVE ESTIMATION OF PROSTATE TISSUE COMPOSITION BASED ON MULTI-PARAMETRIC MRI DATA

(71) Applicants: KONINKLIJKE PHILIPS N.V., EINDHOVEN (NL); The University of Chicago, Chicago, IL (US)

(72) Inventors: AJIT DEVARAJ, Highland Heights, OH (US); GREGORY STANISLAUS KARCZMAR, CRETE, IL (US); AYTEKIN OTO, CHICAGO, IL (US); ARITRICK CHATTERJEE, CHICAGO, IL (US)

(21) Appl. No.: 16/650,470

(22) PCT Filed: Sep. 18, 2018
(86) PCT No.: PCT/EP2018/075117

§ 371 Date: Mar. 25, 2020

Related U.S. Application Data
(60) Provisional application No. 62/563,362, filed on Sep. 26, 2017.
(60) Provisional application No. 62/691,268, filed on Jun. 28, 2018.

(51) Int. Cl. A61B 5/00; G01R 33/56; G01R 33/50; G01R 33/563; A61B 5/055

(52) U.S. Cl. CPC A61B 5/4381; G01R 33/5608; G01R 33/50; A61B 5/7425; G01R 33/56366; A61B 5/004; A61B 5/055; G01R 33/56341

(57) ABSTRACT

A non-transitory storage medium stores instructions readable and executable by at least one electronic processor (20) to perform an imaging method (100). The method includes: obtaining multi-parametric magnetic resonance (MR) imaging data parameterized by a diffusion weighting or perfusion weighting parameter and a magnetization relaxation parameter for a region of interest (ROI) of a patient; determining volume fraction maps of the ROI for each of a plurality of tissue types from the multi-parametric MR imaging data; and controlling a display device (24) to display a tissue composition map comprising or generated from the determined volume fraction maps.

### PRIORITY

This application claims priority to Application Ser. No. U.S. 62/563,362, filed Sep. 26, 2017 and to Application Ser. No. 62/691,268, filed Jun. 28, 2018 which are incorporated herein by reference in their entirety.

### FIELD

The following relates generally to medical imaging arts, glandular tissue monitoring arts, non-invasive tissue monitoring arts, oncology and oncological imaging arts, and related arts.

### BACKGROUND

Prostate cancer is the most common non-cutaneous cancer and second leading cause of death among men in the United States (see, e.g., Siegel R, Naishadham D, Jemal A. Cancer statistics, 2012. C A: A Cancer Journal for Clinicians. 2012; 62(1):10-29; and Johnson L M, Choyke P L, Figg W D, Turkbey B. The Role of MRI in Prostate Cancer Active Surveillance. BioMed Research International. 2014; 2014:6.). A key to effective screening and treatment planning is the knowledge of lesion aggressiveness. The currently accepted approach to assessing the aggressiveness of prostate cancer is the Gleason grading system which is based on tissue architectural changes evaluated on histology. The tissue architectural changes described by the Gleason grading system are directly related to the prostate tissue microstructure i.e. tissue composition (see, e.g., Chatterjee A, Watson G, Myint E, Sved P, McEntee M, Bourne R. Changes in Epithelium, Stroma, and Lumen Space Correlate More Strongly with Gleason Pattern and Are Stronger Predictors of Prostate ADC Changes than Cellularity Metrics. Radiology. 2015; 277(3):751-62; Langer D L, van der Kwast T H, Evans A J, et al. Prostate tissue composition and M R measurements: investigating the relationships between ADC, T2, K(trans), v(e), and corresponding histologic features. Radiology. 2010; 255(2):485-94.; Kobus T, Laak JAWMvd, Maas M C, et al. Contribution of Histopathologic Tissue Composition to Quantitative M R Spectroscopy and Diffusion-weighted Imaging of the Prostate. Radiology. 2015; 278(3):801-11; and Zhao M, Myint E, Watson G, Bourne R. Comparison of conventional histology and diffusion weighted microimaging for estimation of epithelial, stromal, and acinar volumes in prostate tissue. In Proceedings of the 21st Annual Meeting of ISMRM. Salt Lake City, Utah, USA2013; p. 3090).

Magnetic Resonance Imaging (MRI) microscopy of fixed prostate tissue using a preclinical 16T MR has been used to image individual prostatic glands at sufficient resolution (40 μm isotropic) and demonstrated the different MR properties of gland microstructures and changes in these microstructures associated with cancer (see, e.g., Bourne R M, Kurniawan N, Cowin G, et al. Microscopic diffusivity compartmentation in formalin-fixed prostate tissue. Magn Reson Med. 2012; 68(2):614-20; and Bourne R, Kurniawan N, Cowin G, Sved P, Watson G. 16 T diffusion microimaging of fixed prostate tissue: preliminary findings. Magn Reson Med. 2011; 66(1):244-7). However, the spatial resolution of clinical MR scans is far too low to resolve glandular structures as prostate acini are approximately 0.1 mm in diameter (see, e.g., Bourne R. Magnetic resonance microscopy of prostate tissue: How basic science can inform clinical imaging development. Journal of Medical Radiation Sciences. 2013; 60(1):5-10).

Multi-parametric magnetic resonance imaging (mpMRI) is increasingly used in the detection, characterization, and staging of prostate cancer. Most of the current MR biomarkers (e.g. ADC and T2W images) are based on phenomenological models: mono-exponentials, bi-exponentials and kurtosis models. While these phenomenological model based markers are widely accepted for prostate cancer detection clinically, a large number of prostate cancers go undetected, especially smaller significant lesions often go undetected, due to their similarity to the normal gland and benign conditions (see, e.g., Kitzing Y X, Prando A, Varol C, Karczmar G S, Maclean F, Oto A. Benign Conditions That Mimic Prostate Carcinoma: M R Imaging Features with Histopathologic Correlation. RadioGraphics. 2016; 36(1):162-75; and Rosenkrantz A B, Taneja S S. Radiologist, Be Aware: Ten Pitfalls That Confound the Interpretation of Multiparametric Prostate MRI. American Journal of Roentgenology. 2013; 202(1):109-20) and they provide limited information to characterise the lesions. This usually leaves the radiologist with just the legion size and number and maybe longitudinal tracking information to assess cancer aggressiveness. Therefore histological changes remain the reference standard for evaluating cancer aggressiveness.

Studies based on structural models like the ‘vascular, extracellular, and restricted diffusion for cytometry in tumors’ (VERDICT) (see, e.g., Panagiotaki E, Chan R W, Dikaios N, et al. Microstructural Characterization of Normal and Malignant Human Prostate Tissue With Vascular, Extracellular, and Restricted Diffusion for Cytometry in Tumours Magnetic Resonance Imaging. Investigative radiology. 2015; 50(4):218-27) characterize diffusion in three distinct microstructural compartments and may help characterize prostate cancer with good accuracy. However, this model uses increased cellularity as the primary marker for cancer. This is significantly different from histological markers that are known to correlate with prostate cancer (see, e.g., Selnes K M, Vettukattil R, Bertilsson H, et al. Tissue Microstructure Is Linked to MRI Parameters and Metabolite Levels in Prostate Cancer. Frontiers in Oncology. 2016; 6(146)).

A recent study showed that fractional volumes of the prostate gland components: stroma, epithelium, and lumen correlate more strongly with both cancer presence and Gleason grade than ‘cellularity’ metrics. There is an increase epithelial volume with a reduction in luminal and stromal volume in cancers. These changes are more pronounced with increasing Gleason grade. In addition, the tissue components: stroma, epithelium and lumen have distinct diffusivities, and these diffusivities therefore might be used as biomarkers for non-invasive prostate cancer detection. Two recent studies showed that luminal volume estimated using a bi-exponential model to fit multi-echo T2-weighted images, or Luminal Water Imaging (LWI) could be used for detection of prostate cancer (see, e.g., Sabouri S, Fazli L, Chang S D, et al. MR measurement of luminal water in prostate gland: Quantitative correlation between MRI and histology. Journal of Magnetic Resonance Imaging. 2017:n/a-n/a; and Sabouri S, Chang S D, Savdie R, et al. Luminal Water Imaging: A New MR Imaging T2 Mapping Technique for Prostate Cancer Diagnosis. Radiology. 2017; 0(0):161687).

The following discloses new and improved systems and methods to overcome these problems.

### SUMMARY

In one disclosed aspect, a non-transitory storage medium stores instructions readable and executable by at least one electronic processor to perform an imaging method. The method includes: obtaining multi-parametric magnetic resonance (MR) imaging data parameterized by a diffusion weighting or perfusion weighting parameter and a magnetization relaxation parameter for a region of interest (ROI) of a patient; determining volume fraction maps of the ROI for each of a plurality of tissue types from the multi-parametric MR imaging data; and controlling a display device to display a tissue composition map comprising or generated from the determined volume fraction maps.

In another disclosed aspect, a medical imaging workstation includes a processor configured to generate from an MRI image a prostatic tissue composition map (pTCM) by transforming a multi-dimensional array of MRI data using a model to non-invasively evaluate volume fractions, ADC's, and T2's of prostate tissue components.

In another disclosed aspect, an imaging method includes: obtaining multi-parametric magnetic resonance (MR) imaging data parameterized by a diffusion weighting parameter and a magnetization relaxation parameter for a region of interest (ROI) of a patient; determining volume fraction maps of the ROI for each of a plurality of tissue types from the multi-parametric MR imaging data; and controlling a display device to display a tissue composition map comprising or generated from the determined volume fraction maps.

One advantage resides in non-invasively detecting potential tumors in a target organ of a patient.

Another advantage resides in providing for non-invasive grading of cancer.

Another advantage resides in detecting potential tumors in the target organ without obtaining a biopsy.

Another advantage resides in providing for simultaneous imaging of the volume fractions of a plurality of different tissue types.

Another advantage resides in measuring properties of each compartment based on the ADC and T2 of each compartment.

Another advantage resides in detecting and measuring an additional compartment of water with a restricted diffusion and a long T2 value that is associated with cancer.

A given embodiment may provide none, one, two, more, or all of the foregoing advantages, and/or may provide other advantages as will become apparent to one of ordinary skill in the art upon reading and understanding the present disclosure.

### BRIEF DESCRIPTION OF THE DRAWINGS

The disclosure may take form in various components and arrangements of components, and in various steps and arrangements of steps. The drawings are only for purposes of illustrating the preferred embodiments and are not to be construed as limiting the disclosure.

FIG. 1 diagrammatically shows an illustrative imaging system in accordance with one aspect;

FIG. 2 shows an exemplary flow chart operation of the system of FIG. 1;

FIG. 3 shows an example schematic of a map generated by the system of FIG. 1; and

FIGS. 4 and 5 show results of an imaging method performed by the system of FIG. 1.

### DETAILED DESCRIPTION

MRI prostate cancer imaging can be used preparatory to surgery (i.e. MRI planning images), or for screening, guiding biopsies, cancer grading, non-invasive monitoring, or so forth. T2 imaging or diffusion weighted imaging (DWI) are common imaging techniques for this purpose. However, these techniques have certain deficiencies, due to interrelatedness of the T2 and DWI contrast mechanisms.

In embodiments disclosed herein, a parameterized model of the MRI signal is developed, which defines various tissue types (stroma, epithelium, and lumen in the case of the prostate or some other glands; the tissue types are sometimes referred to as tissue compartments). The model parameters include volume fraction, T2, and apparent diffusion coefficient (ADC) for each tissue type. For the stroma/epithelium/lumen model, this results in nine model parameters. By acquiring multi-parametric magnetic resonance (MR) imaging data, such as, for example, a 3×3 matrix of different (TE,b-value) pairs (or, more generally, 9 different (TE,b-value) pairs), nine equations can be created by applying the model to predict the MR signal for each (TE,b-value) pair. Additionally, the partial volumes must sum to unity, providing a tenth equation. Thus, for each voxel there are nine unknowns and ten equations. This model applied to the 3×3 multi-parametric MR imaging data can therefore be solved for the volume fractions of the stroma, epithelium, and lumen at each voxel. The resulting volume fraction values for each voxel may be displayed as a prostatic tissue composition map (pTCM). This may be displayed as three maps (one for each tissue type) or as a composite map, e.g. using color coding. Larger matrices have also been used, but this increases acquisition time.

These techniques can be generalized to other parameter pairs (magnetization relaxation parameter, DWI parameter besides (TE,b-value)). For example, b-value adjustment can be obtained by varying the diffusion gradient amplitude or the diffusion time between gradients. The magnetization relaxation parameter may for example be the echo time (TE), the flip angle, the time-to-repeat (TR), pulses that induce magnetization transfer, or other parameters. Additionally, in some embodiments the DWI parameterization is replaced by Diffusion Contrast Enhanced (DCE) imaging parameterization, i.e. the multi-parametric MR imaging data may be parameterized using (magnetization relaxation parameter, DCE parameter) pairs.

The disclosed systems and methods can be applied to any gland-based organ having stroma, epithelium, and lumen tissue types (for example, prostate, colorectal, breast, pancreas, or thyroid), and even more generally to other organs assuming suitable tissue type definitions are employed. For example, the liver could be similarly analyzed using the following tissue types: bioduct; hepatocellular; stroma; and vasculature.

In embodiments disclosed herein, all nine parameters obtained from a three compartment model are solved independently at each voxel. In variant envisioned approaches, various correlations may be leveraged as constraints or additional limitations, such as a constraint that the T2 and/or ADC parameters are slowly varying (or even constant) over the volume. Additionally, it is contemplated to acquire more than 9 different (TE,b-value) combinations, and/or for those combinations to not form a 3×3 matrix.

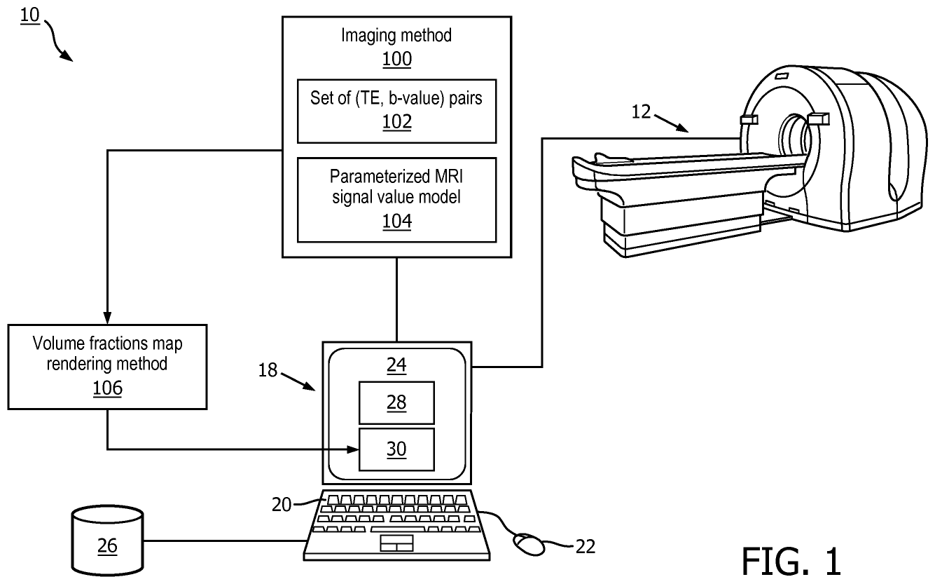

With reference to FIG. 1, an imaging system or device 10 is shown. As shown in FIG. 1, the device 10 includes a magnetic resonance (MR) imaging device 12 configured to acquire MR imaging data using a DWI sequence with diffusion gradients designed for a particular b-value (or, in alternative embodiment, using a DCE which can be used to produce parameters such as Ktrans (computed from the images as an imaging biomarker), ye, maximum rate of enhancement, and time of initial enhancement.

The device 10 also includes a medical imaging workstation 18 comprising a computer or other electronic data processing device with typical components, such as at least one electronic processor 20, at least one user input device (e.g., a mouse, a keyboard, a trackball, and/or the like) 22, and a display device 24. It should be noted that these components can be variously distributed. For example, the electronic processor 20 may include a local processor of a workstation terminal and the processor of a server computer that is accessed by the workstation terminal. In some embodiments, the display device 24 can be a separate component from the computer 18. The workstation 18 can also include one or more databases or non-transitory storage media 26. The non-transitory storage media 26 may, by way of non-limiting illustrative example, include one or more of a magnetic disk, RAID, or other magnetic storage medium; a solid state drive, flash drive, electronically erasable read-only memory (EEROM) or other electronic memory; an optical disk or other optical storage; various combinations thereof; or so forth. The display device 24 is configured to display a graphical user interface (GUI) 28 including one or more fields to receive a user input from the user input device 22. A generated map 30 of the volume fractions of the various tissue types (described in more detail below) can be displayed on the display device 24.

The system 10 comprises the medical imaging workstation 18 with the processor 20 configured to generate from an MRI image a prostatic tissue composition map (pTCM) by transforming a multi-dimensional array of MRI data using a model to non-invasively evaluate volume fractions, ADC's, and T2's of prostate tissue components. In illustrative examples, the tissue components comprise stroma, epithelium and lumen which is a suitable tissue compartmentalization model for the prostate and for some other glands or glandular tissue structures such as the rectum and/or colon, breast, pancreas, or thyroid gland.

The system 10 is configured to perform an imaging method or process 100 which acquires multi-parametric MR imaging data for a set of (magnetization relaxation parameter, DWI or DCE parameter) pairs 102, and more particularly for a set of (TE,b-value) pairs 102 in the example for prostate cancer imaging of FIG. 1, and applies a parameterized MRI signal value model 104 to the acquired multi-parametric MR imaging data to generate volume fraction values for the various tissue types of the model 104 (e.g. for the stroma, epithelial, and lumen tissue types in the illustrative prostate cancer imaging case) for each voxel of a region of interest (ROI), thereby forming volume fraction maps for the various tissue types. A volume fractions map rendering method 106 generates a one or more map renderings, i.e. map(s) 30, of the volume fractions mapping data output by the imaging method or process 100, which is or are suitably displayed on the display 24. A non-transitory storage medium (e.g., non-transitory storage media 26) stores instructions which are readable and executable by the at least one electronic processor 20 of the workstation 18 and to perform disclosed operations including performing the treatment planning method or process 100 and the volume fractions map rendering method 106. In some examples, one or both methods 100, 106 may be performed at least in part by cloud processing.

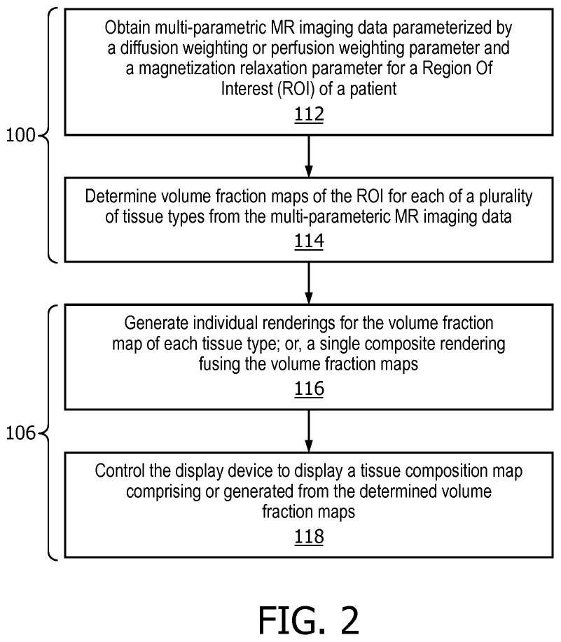

With reference to FIG. 2, an illustrative embodiment of the treatment planning method 100 and the rendering method 106 is diagrammatically shown as a flowchart. At 112, the at least one electronic processor 20 is programmed to acquire multi-parametric MR imaging data parameterized by a diffusion weighting or perfusion weighting parameter values and different magnetization relaxation parameter values (that is, for the set of parameter value pairs 102, see FIG. 1) for a region of interest (ROI) of a patient. In some examples, the at least one of a diffusion weighting or perfusion parameter includes a diffusion weighting parameter, and the diffusion weight parameter comprises one of a b-value, a diffusion time, or a diffusion gradient. In other examples, the at least one of a diffusion weighting or perfusion parameter value includes a perfusion weighting parameter. In further examples, the at least one magnetization relaxation parameter comprises one of echo time (T_{E}) or flip angle. In some examples embodiments, the at least one magnetization relaxation parameter value comprises echo time (T_{E}) and the diffusion weighting parameter value comprises a b-value.

At 114, the at least one electronic processor 20 is programmed to determine volume fraction maps of the ROI for each of a plurality of tissue types from the multi-parametric MR imaging data using the parameterized MRI signal value model 104 (see FIG. 1). For example, the plurality of tissue types includes a stroma tissue type, an epithelial tissue type, and a lumen tissue type. In some examples, the determining includes solving a system of equations for each voxel to determine the volume fraction values for each tissue type of the plurality of tissue types, where the system of equations for each voxel includes an equation for each value pair of: (i) the diffusion weighting or perfusion weighting parameter, and the (ii) magnetization relaxation parameter, in the multi-parametric MR imaging data. In addition, the system of equations for each voxel further includes an equation requiring that the sum of the volume fractions equals one. Other constraints or limitations may optionally be incorporated. By this process, the volume fractions along with any other variables of the model 104 (e.g., the T₂ values and apparent diffusion coefficient (ADC) values in the illustrative example) are determined for each tissue type of the plurality of tissue types.

In some embodiments, the multi-parametric MR imaging data includes MR imaging data acquired for at least nine (T_{E},b-value) parameter value pairs. To determine the volume fraction maps, the determining of the volume fraction maps includes solving a system of equations at each voxel for at least the volume fraction of each tissue type. The system of equations includes, for each (T_{E},b-value) parameter value pair, an equation relating MR signal of the voxel acquired with the (T_{E},b-value) parameter value pair to a weighted sum of signal components for each tissue type functionally depending on relaxation time T₂ and ADC of the tissue type with each signal component weighted by the volume fraction of the tissue type. The equation can also be expressed as solving equations at each voxel for at least the volume fraction V_{T}_{ᵢ} of each tissue type Tᵢ∈{T} where {T} denotes the set of the plurality of tissue types, wherein the system of equations includes, for each (T_{E},b-value) parameter value pair, The equation can be expressed as Equation 1:

$$\frac{S}{{S}_{0}} = {\sum }_{{T}_{i} ∈ \{ T \}} {V}_{{T}_{i}} exp ( - \frac{{T}_{E}}{{T}_{{2}_{{T}_{i}}}} ) exp ( - ( b \times A D {C}_{{T}_{i}} ) \qquad ( 1 )$$

where S is the MR signal at the voxel, S₀ is a constant, T_{E} and b are the echo time T_{E} and b-value, respectively, of the (T_{E},b-value) parameter value pair and

$${T}_{{2}_{{T}_{i}}}$$

and ADC_{T}_{ᵢ} denote the relaxation time T₂ and ADC of the tissue type Tᵢ. Thus, as demonstrated in Equation 1, the system of equations at each voxel is solved for the volume fraction of each tissue type and relaxation time T₂ and ADC of each tissue type (e.g.,

$${T}_{{2}_{{T}_{i}}}$$

### and ADC_{T}_{ᵢ}).

Furthermore, the system of equations at each voxel further includes an equation requiring that the sum of the volume fractions equals one. This can be expressed as Equation 2:

Σ_{T}_{ᵢ}_{∈{T}}V_{T}_{ᵢ}=1 (2)

requiring that the sum of the volume fractions equals one. Additional or other equations and/or constraints are contemplated to provide a more overdetermined and/or constrained set of equations. For example, the T₂ and/or ADC values may be constrained to a physically realistic range, using either hard constraints (e.g., not allowing the fitted T₂ to go outside of some specified physically realistic range) or soft constraints (e.g., penalizing T₂ by an increasingly large penalty value the further the fitted T₂ strays from a prior-known “typical” value of T₂ for the tissue). Another contemplated constraint is to penalize excessive spatial variation—this approach can improve smoothness of the fit, albeit at a cost of greater computational complexity due to the equations for neighboring voxels being interdependent. As another contemplated approach, T₂ and/or ADC for a particular tissue may be set to a constant “typical” value for the tissue, which simplifies computational complexity and may smooth the data, but at a possible cost of some increased error in the determined volume fractions.

At 116, the at least one electronic processor 20 is programmed to generate individual renderings for the volume fraction map of each tissue type. This can be done, for example, by rendering each volume fraction map (one for stroma, one for epithelium, and one for lumen, in the illustrative prostate example) as a grayscale or color-coded rendering of the volume fraction values of the voxels of the ROI. Alternatively, operation 116 may generate a single composite rendering that fuses the volume fraction maps. One approach for this is to employ a red-green-blue (RGB) color model (or some other chosen three-component color model) and to set the intensity of each color component of each voxel of the rendering to the voxel volume fraction of a corresponding tissue type. Optionally, both individual tissue type renderings and a single composite rendering may be generated at operation 116.

At 118, the at least one electronic processor 20 is programmed to control the display device 24 to display the rendering(s) generated at operation 116, e.g. to display a tissue composition map comprising or generated from the determined volume fraction maps. In one example, the display device 24 is controlled to display the tissue composition map as a display of the volume fraction map for each tissue type of the plurality of tissue types. In another example, the display device 24 is controlled to display the tissue composition map as a single composition map fusing the volume fraction maps for the plurality of tissue types.

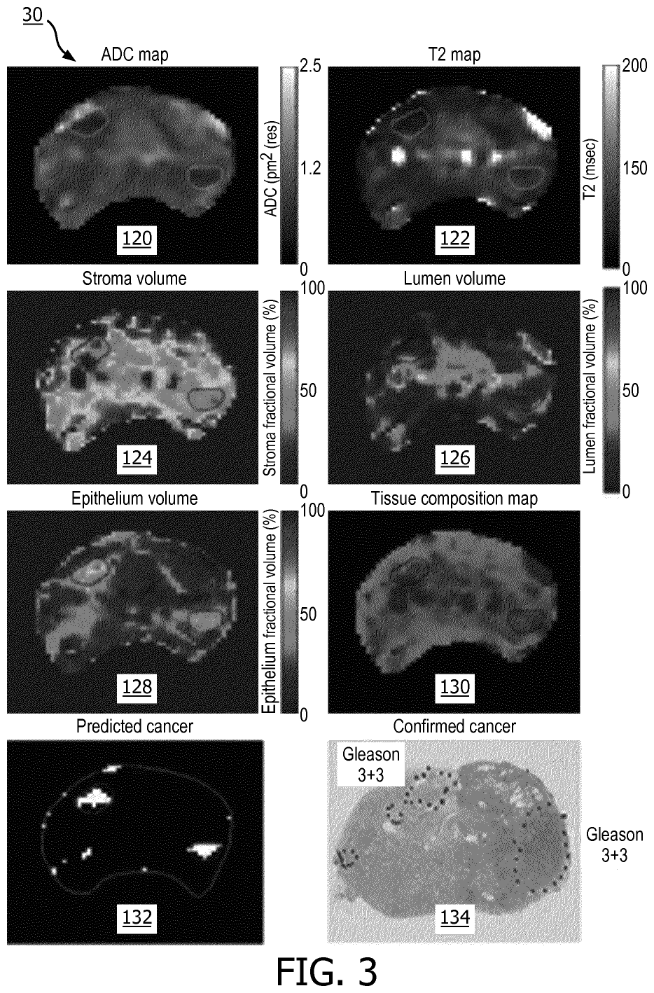

FIG. 3 shows several examples of composite maps 30 for display on the display device 24. The maps 30 shown in FIG. 3 include: the ADC map 120 and the T₂ map 122; the individual volume fraction maps, namely the stroma volume fraction map 124, the lumen volume fraction map 126, and the epithelium volume fraction map 128; a prostatic tissue composition map (pTCM) 130; and a predicted cancer map 132 derived from the volume fraction maps 124, 126, 128 and a corresponding map 134 showing confirmed cancer on, for example, hematoxylin and eosin stained tissue.

### Example

Advantageously, the disclosed method 100 can be validated against quantitative histology. Specifically, MRI provides measurements of epithelial, stromal, and lumen volume fractions that closely agree with quantitative histology. This demonstrates for the first time the use of in vivo MRI to measure volume fractions of epithelia, stroma, and lumen non-invasively (see below and in Chatterjee, A., Bourne, R. M., Karczmar, G. S., Oto, A. et al, Diagnosis of Prostate Cancer with Noninvasive Estimation of Prostate Tissue Composition by using Hybrid Multidimensional MR imaging: A Feasibility Study., Radiology, 2018).

If it is assumed that ADC and T2 are independent parameters, then components with different ADC's may be identified from modelling diffusion data and components with different T2's may be identified from spin echo measurements (see, e.g., Stor∪s TH, Gjesdal K-I, Gadmar ØB, Geitung JT, Klow N-E. Prostate magnetic resonance imaging: Multiexponential T2 decay in prostate tissue. Journal of Magnetic Resonance Imaging. 2008; 28(5):1166-72; and Kjaer L, Thomsen C, Iversen P, Henriksen O. In vivo estimation of relaxation processes in benign hyperplasia and carcinoma of the prostate gland by magnetic resonance imaging. Magnetic resonance imaging. 1987; 5(1):23-30). However, in this approach it is not known whether the different components identified by diffusion and T2 measurements have a direct one-to-one correspondence (see, e.g., Bourne R, Panagiotaki E. Limitations and Prospects for Diffusion-Weighted MRI of the Prostate. Diagnostics. 2016; 6(2):21, and Wange, S., Oto, A., Karczmar, G. S. eta 1, Hybrid Multidimentional T2 and diffusion-weighted MRI for prostate cancer detection, J Magnetic Resonance Imaging, 2014).

For example, by co-registering in vivo MRI with Hematoxylin and Eosin stained whole mount prostatic tissue,—the volume fractions of epithelium, stroma, and lumens measured by in vivo HM-MRI agree closely with gold standard histology. Specifically, patients (n=8, age=62 years, PSA=15.0 ng/ml) with biopsy confirmed PCa underwent preoperative 3T prostate MRI prior to undergoing radical prostatectomy. Axial images using HM-MRI were acquired with all combinations of TE=57, 70, 150, 200 ms and b-values of 0, 150, 750, 1500 s/mm², resulting in a 4×4 array of data associated with each voxel. Volumes of tissue components-stroma, epithelium and lumen were calculated by fitting the hybrid data to a three compartment signal model, with distinct, paired ADC and T2 values associated with each compartment. Quantitative histology was performed to calculate volumes of tissue components on rectangular ROIs from region corresponding to the MR ROIs (n=16, 8 PCa, 8 normal tissue) using Image Pro Premier.

There was no significant difference (paired t-test) in prostate tissue composition: stroma (43.8±11.8 vs 41.6±8.7, p=0.20), epithelium (34.2±18.1 vs 36.7±15.3, p=0.07) and lumen (22.0±13.1 vs 21.7±10.9, p=0.80) measured using HM-MRI and quantitative histology. There was excellent Pearson correlation (overall=0.94, stroma=0.84, epithelium=0.97, lumen=0.93, p<0.05) between prostate tissue composition measured using these methods. ROC analysis showed high area under the curve using HM-MRI (epithelium=0.89, lumen=1.00) and histology (epithelium=0.91, lumen=0.93) results, suggesting PCa can be differentiated from normal tissue estimated non-invasively by HM-MRI due to increased epithelium and reduced luminal volume in PCa compared to normal tissue.

For example, when ADC and T2 are measured using a spin echo module with EPI readout, ADC changes significantly as a function of TE, and T2 changes significantly as a function of b-value. Hybrid Multi-dimensional MRI (HM-MRI) imaging measures the change in ADC and T2 as a function of TE and b-value, respectively, and uses these changes as a source of information about the underlying tissue microstructure. As a result, HM-MRI can provide quantitative maps of prostatic tissue composition by exploiting the coupled T2 and ADC values associated with each tissue component.

This pulse sequence was used to acquire images with a set of TEs of (typical values are 47, 75 and 100 msec). At each TE images were acquired with a range of b-values (typical values are 0, 750, and 1500 s/mm²), resulting in a multi-dimensional array of data (if all pair combinations of the foregoing TEs and b-values are acquired, this would result in a 3×3 array) of parameter pair values:

$$[ \begin{matrix}( 47 msec , 0 s / {mm}^{2} ) & ( 47 msec , 750 s / {mm}^{2} ) & ( 47 msec , 1550 s / {mm}^{2} ) \\ ( 75 msec , 0 s / {mm}^{2} ) & ( 75 msec , 750 s / {mm}^{2} ) & ( 75 msec , 1550 s / {mm}^{2} ) \\ ( 100 msec , 0 s / {mm}^{2} ) & ( 100 msec , 750 s / {mm}^{2} ) & ( 100 msec , 750 s / {mm}^{2} )\end{matrix}$$

associated with each image voxel. HM-MRI images were acquired in the axial plane and oriented perpendicular to the rectal wall, as guided by sagittal images. Fat saturation was performed using spectrally adiabatic inversion recovery (SPAIR).

The MR signal from prostate tissue is modelled as unmixed pools of water in three tissue components: stroma, epithelium and lumen. Tissue component volumes were calculated on a voxel by voxel basis by fitting Equation 3 (corresponding to Equation 1 for the specific three-tissue-type prostate model):

$$\frac{s}{{s}_{0}} = {V}_{stroma} exp ( - T E / {T}_{{2}_{stroma}} ) exp ( - b \times A D {C}_{strom a} ) + {V}_{epithelium} exp ( - T E / {T}_{{2}_{epithelium}} ) exp ( - b \times A D {C}_{epithelium} ) + {V}_{l u m e n} exp ( - T E / {T}_{{2}_{l u m e n}} ) exp ( - b \times A D {C}_{l u m e n} ) \qquad ( 3 )$$

where Vₛₜᵣₒₘₐ, Vₑₚᵢₜₕₑₗᵢᵤₘ, and Vₗᵤₘₑₙ amen are the volume fractions of each compartment, T₂_{ₛₜᵣₒₘₐ}, T₂_{ₑₚᵢₜₕₑₗᵢᵤₘ}, and T₂_{ₗᵤₘₑₙ} are the T₂'s for each compartment and ADCₛₜᵣₒₘₐ, ADCₑₚᵢₜₕₑₗᵢᵤₘ and ADCₗᵤₘₑₙ are the ADC's for each compartment.

In addition, only three tissue components (stroma, epithelium and lumen) are present (e.g. the contribution from blood was not estimated), so that the sum of volume fraction of all three tissue components is one, as expressed in Equation 4 (corresponding to Equation (2) for the specific three-tissue-type prostate model):

Vₛₜᵣₒₘₐ+Vₑₚᵢₜₕₑₗᵢᵤₘ+ₗᵤₘₑₙ=1 (4)

Anterior Fibromuscular Stroma (AFMS) has dense stromal fibers and is therefore assumed to have lower ADC and T₂ value than stroma in the rest of the prostate (see, e.g., Amin M, Khalid A, Tazeen N, Yasoob M. Zonal Anatomy of Prostate. Annals of King Edward Medical University. 2011; 16(3); Ward E, Baad M, Peng Y, et al. Multi-parametric M R imaging of the anterior fibromuscular stroma and its differentiation from prostate cancer. Abdom Radiol. 2016:1-9; and McNeal J E. Normal histology of the prostate. Am J Surg Pathol. 1988; 12(8):619-33). The lower limits of stromal ADC and T₂ were determined based on the ADC's (0.7 μm²/ms) and T₂'s (40 ms) measured in the AFMS (see, e.g., Niaf E, Lartizien C, Bratan F, et al. Prostate Focal Peripheral Zone Lesions: Characterization at Multiparametric M R Imaging—Influence of a Computer-aided Diagnosis System. Radiology. 2014; 271(3):761-9; Baur A D, Maxeiner A, Franiel T, et al. Evaluation of the prostate imaging reporting and data system for the detection of prostate cancer by the results of targeted biopsy of the prostate. Invest Radiol. 2014; 49(6):411-20; Hoeks C M A, Vos E K, Bomers J G R, Barentsz J O, Hulsbergen-van de Kaa C A, Scheenen T W. Diffusion-Weighted Magnetic Resonance Imaging in the Prostate Transition Zone: Histopathological Validation Using Magnetic Resonance-Guided Biopsy Specimens. Investigative Radiology. 2013; 48(10):693-701; Akin O, Sala E, Moskowitz C S, et al. Transition Zone Prostate Cancers: Features, Detection, Localization, and Staging at Endorectal MR Imaging. Radiology. 2006; 239(3):784-92; Shiraishi T, Chikui T, Yoshiura K, Yuasa K. Evaluation of T(2) values and apparent diffusion coefficient of the masseter muscle by clenching. Dentomaxillofacial Radiology. 2011; 40(1):35-41; and Langer D L, van der Kwast T H, Evans A J, et al. Intermixed normal tissue within prostate cancer: effect on M R imaging measurements of apparent diffusion coefficient and T2—sparse versus dense cancers. Radiology. 2008; 249(3):900-8).

Cancer is associated with reduced ADC and T₂. While cancer is primarily composed of epithelium, ‘pure’ epithelium will likely have even lower ADC and T₂ values than cancer lesions (4). Therefore, the ADC (e.g., 0.7 μm²/ms) and T₂ (e.g., 70 ms) in high grade values obtained from high grade cancer were used as the upper limit for the epithelium component.

The upper limit for fitting luminal ADC and were set lower than the values of pure water at 37° C. Luminal fluid should have lower ADC and T₂ due to the presence of proteins.

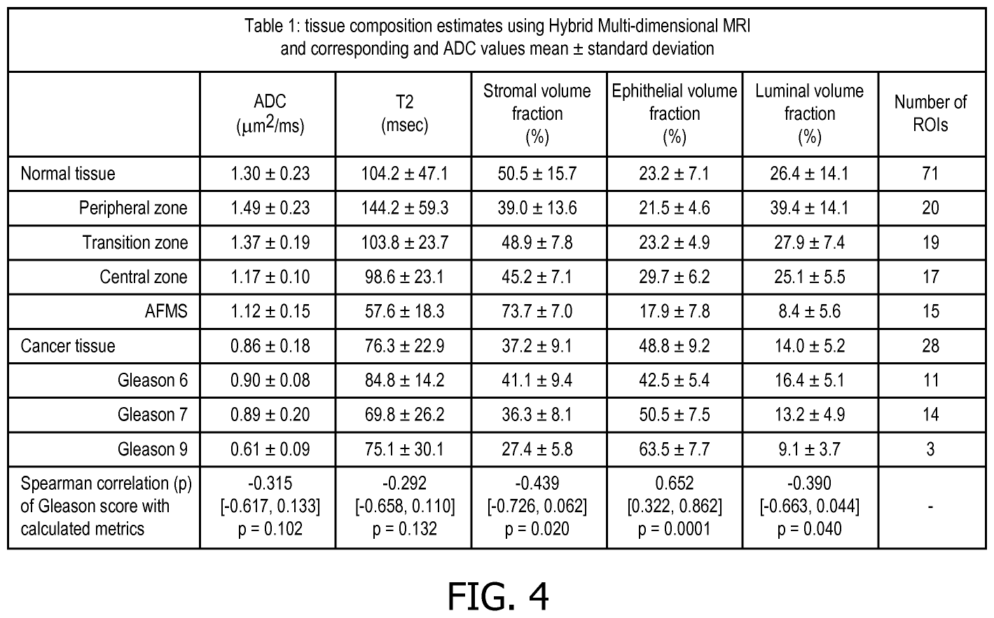

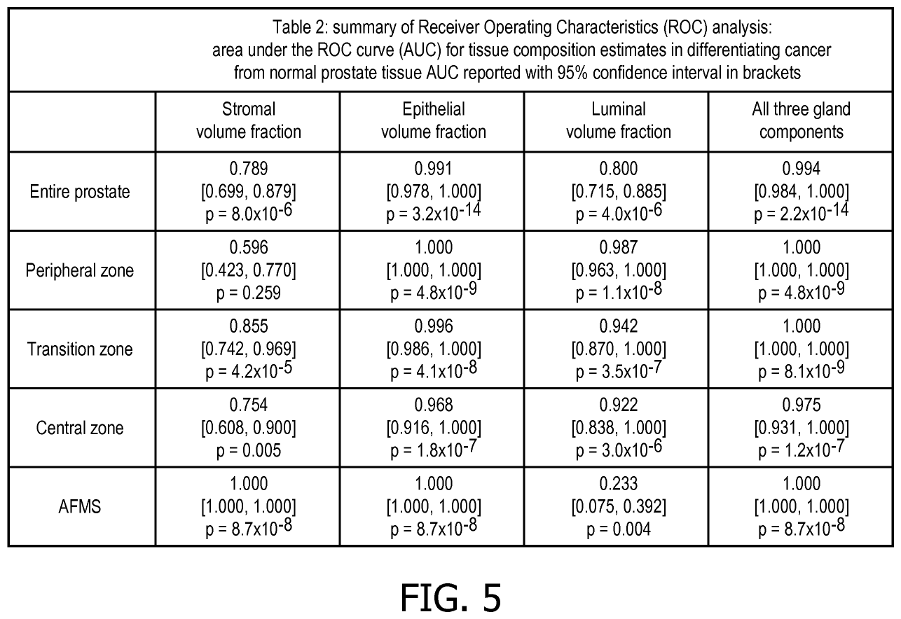

FIGS. 4 and 5 show results from a pilot study. The shaded values show the ability of the computed pTCM values to detect cancer and predict cancer aggressiveness.

The disclosure has been described with reference to the preferred embodiments. Modifications and alterations may occur to others upon reading and understanding the preceding detailed description. It is intended that the disclosure be construed as including all such modifications and alterations insofar as they come within the scope of the appended claims or the equivalents thereof.

1. A non-transitory storage medium storing instructions readable and executable by at least one electronic processor to perform an imaging method, the method comprising:
obtaining multi-parametric magnetic resonance (MR) imaging data parameterized by a diffusion weighting or perfusion weighting parameter and a magnetization relaxation parameter for a region of interest (ROI) of a patient;
determining volume fraction maps of the ROI for each of a plurality of tissue types from the multi-parametric MR imaging data; and
controlling a display device to display a tissue composition map comprising or generated from the determined volume fraction maps.

2. The non-transitory storage medium of claim 1, wherein the at least one of a diffusion weighting or perfusion parameter includes a diffusion weighting parameter, and the diffusion weight parameter comprises one of a b-value, a diffusion time, or a diffusion gradient.

3. The non-transitory storage medium of claim 1, wherein the at least one of a diffusion weighting or perfusion parameter value includes a perfusion weighting parameter.

4. The non-transitory storage medium of claim 1, wherein the at least one magnetization relaxation parameter comprises one of echo time (T_{E}) or flip angle.

5. The non-transitory storage medium of claim 1, wherein the at least one magnetization relaxation parameter value comprises echo time (T_{E}) and the diffusion weighting parameter value comprises a b-value.

6. The non-transitory storage medium of claim 5, wherein:
the multi-parametric magnetic resonance (MR) imaging data includes MR imaging data acquired for at least nine (T_{E},b-value) parameter value pairs, and
the determining of the volume fraction maps includes solving a system of equations at each voxel for at least the volume fraction of each tissue type wherein the system of equations includes, for each (T_{E},b-value) parameter value pair, an equation relating MR signal of the voxel acquired with the (T_{E},b-value) parameter value pair to a weighted sum of signal components for each tissue type functionally depending on relaxation time T₂ and apparent diffusion coefficient (ADC) of the tissue type with each signal component weighted by the volume fraction of the tissue type.

7. The non-transitory storage medium of claim 6, wherein the system of equations at each voxel further includes an equation requiring that the sum of the volume fractions equals one.

8. The non-transitory storage medium of claim 6, wherein the system of equations at each voxel is solved for the volume fraction of each tissue type and relaxation time T₂ and ADC of each tissue type.

9. The non-transitory storage medium of claim 5, wherein:
the multi-parametric magnetic resonance (MR) imaging data includes MR imaging data acquired for at least nine (T_{E},b-value) parameter value pairs, and
the determining of the volume fraction maps includes solving a system of equations at each voxel for at least the volume fraction V_{T}_{ᵢ} of each tissue type Tᵢ∈{T} where {T} denotes the set of the at least three tissue types, wherein the system of equations includes, for each (T_{E},b-value) parameter value pair, the equation:
$$\frac{S}{{S}_{0}} = {\sum }_{{T}_{i} ∈ \{ T \}} {V}_{{T}_{i}} exp ( - \frac{{T}_{E}}{{T}_{{2}_{{T}_{i}}}} ) exp ( - ( b \times A D {C}_{{T}_{i}} )$$
where S is the MR signal at the voxel, S₀ is a constant, T_{E} and b are the echo time T_{E} and b-value, respectively, of the (T_{E},b-value) parameter value pair and
$${T}_{{2}_{{T}_{i}}}$$
and ADC_{T}_{ᵢ} denote the relaxation time T2 and ADC of the tissue type Tᵢ.

10. The non-transitory storage medium of claim 9, wherein the system of equations at each voxel further includes the equation Σ_{T}_{_{i∈{T}}}V_{T}_{ᵢ}=1 requiring that the sum of the volume fractions equals one.

11. The non-transitory storage medium of claim 9, wherein the system of equations at each voxel is solved for the volume fraction V_{T}_{ᵢ} of each tissue type and for the relaxation time
$${T}_{{2}_{{T}_{i}}}$$
and ADC_{T}_{ᵢ} of each tissue type.

12. The non-transitory storage medium of claim 1, wherein the determining comprises:
solving a system of equations for each voxel to determine the volume fraction values for each tissue type of the plurality of tissue types where the system of equations for each voxel includes an equation for each value pair of the diffusion weighting or perfusion weighting parameter and the magnetization relaxation parameter in the multi-parametric MR imaging data.

13. The non-transitory storage medium of claim 12, wherein the system of equations for each voxel further includes an equation requiring that the sum of the volume fractions equals one.

14. The non-transitory storage medium of claim 1, wherein the determining further includes:
determining T₂ values and apparent diffusion coefficient (ADC) values for each tissue type of the plurality of tissue types.

15. The non-transitory storage medium of claim 1, wherein the plurality of tissue types includes a stromal tissue type, an epithelial tissue type, and a lumen tissue type;
wherein the volume fractions for the stromal tissue type, the epithelial tissue type, and the lumen tissue type are measured non-invasively by in vivo pTCM and compared with quantitative analysis of whole mount hematoxylin and eosin stained tissue.

16. The non-transitory storage medium of claim 1, wherein the display device is controlled to display the tissue composition map comprising at least one of:
display of the volume fraction map for each tissue type of the plurality of tissue types; or
display of a single composition map fusing the volume fraction maps for the at least three tissue types.

17. A medical imaging workstation comprising a processor configured to generate from an MRI image a prostatic tissue composition map (pTCM) by transforming a multi-dimensional array of MRI data using a model to non-invasively evaluate volume fractions, ADC's, and T2's of prostate tissue components.

18. The medical imaging workstation of claim 17 wherein the tissue components comprise stroma, epithelium and lumen.

19. The medical imaging workstation of claim 18 wherein:
the multi-dimensional array of MRI data is parameterized by echo time T_{E} and b-value and comprises MR imaging data acquired for at least nine (T_{E},b-value) parameter value pairs; and
the pTCM map is generated by solving a system of equations at each voxel for the volume fractions Vₛₜᵣₒₘₐ, Vₑₚᵢₜₕₑₗᵢᵤₘ, and Vₗᵤₘₑₙ of the stroma, epithelium, and lumen respectively and for the relaxation times T₂_{ₛₜᵣₒₘₐ}, T₂_{ₑₚᵢₜₕₑₗᵢᵤₘ}, and T₂_{ₗᵤₘₑₙ} of the stroma, epithelium, and lumen respectively and for the apparent diffusion coefficients ADCₛₜᵣₒₘₐ, ADCₑₚᵢₜₕₑₗᵢᵤₘ, and ADCₗᵤₘₑₙ of the stroma, epithelium, and lumen respectively wherein the system of equations includes, for each (T_{E},b-value) parameter value pair, the equation:
$$\frac{S}{{S}_{0}} = {V}_{s t r o m a} exp ( - \frac{{T}_{E}}{{T}_{{2}_{s t τ o m a}}} ) exp ( - ( b \times A D {C}_{s t r o m a} ) + {V}_{epithelium} exp ( - \frac{{T}_{E}}{{T}_{{2}_{epithelium}}} ) exp ( - ( b \times A D {C}_{e p i t h e l i u m} ) + {V}_{l u m e n} exp ( - \frac{{T}_{E}}{{T}_{{2}_{lumen}}} ) exp ( - ( b \times A D {C}_{l u m e n} ) \qquad$$
Where S is the MR signal at the voxel, S₀ is a constant, and T_{E} and b are the echo time T_{E} and b-value, respectively, of the (T_{E},b-value) parameter value pair.

20. An imaging method, comprising:
obtaining multi-parametric magnetic resonance (MR) imaging data parameterized by a diffusion weighting parameter and a magnetization relaxation parameter for a region of interest (ROI) of a patient;
determining volume fraction maps of the ROI for each of a plurality of tissue types from the multi-parametric MR imaging data; and
controlling a display device to display a tissue composition map comprising or generated from the determined volume fraction maps.
